(19) United States
(12) Patent Application Publication
(10) Pub. No.: US 2007/0078374 A1
(43) Pub. Date: Apr. 5, 2007

(54) Iontophoretic delivery of vesicle-encapsulated active agents

(75) Inventor: Gregory A. Smith, Sammamish, WA (US)

Correspondence Address: SEED INTELLECTUAL PROPERTY LAW GROUP PLLC, 701 FIFTH AVE, SUITE 5400, SEATTLE, WA, 98104, US

(73) Assignee: Transcutaneous Technologies Inc., Tokyo (JP)

(21) Appl. No.: 11/540,949

(22) Filed: Sep. 28, 2006

Related U.S. Application Data
(60) Provisional application No. 60/722,298, filed on Sep. 30, 2005.

(51) Int. Cl. A61N 1/30

(52) U.S. Cl. 604/20

(57) ABSTRACT

An iontophoresis device is provided for the delivery of active agents encapsulated in vesicles such as transferosomes and niosomes to a biological interface such as skin or mucous membranes.

### CROSS-REFERENCE TO RELATED APPLICATION

This application claims the benefit under 35 U.S.C. § 119(e) of U.S. Provisional Patent Application No. 60/722,298, filed Sep. 30, 2005.

### BACKGROUND OF THE INVENTION

1. Field of the Invention

This disclosure generally relates to the field of iontophoresis, and more particularly to the delivery of active agents such as therapeutic agents or drugs under the influence of electromotive force.

2. Description of the Related Art

Iontophoresis employs an electromotive force to transfer an active agent such as an ionic drug or other therapeutic agent to a biological interface, for example skin or mucus membrane.

Iontophoresis devices typically include an active electrode assembly and a counter electrode assembly, each coupled to opposite poles or terminals of a power source, for example a chemical battery. Each electrode assembly typically includes a respective electrode element to apply an electromotive force. Such electrode elements often comprise a sacrificial element or compound, for example silver or silver chloride.

The active agent may be either cation or anion, and the power source can be configured to apply the appropriate voltage polarity based on the polarity of the active agent. Iontophoresis may be advantageously used to enhance or control the delivery rate of the active agent. As discussed in U.S. Pat No. 5,395,310, the active agent may be stored in a reservoir such as a cavity. Alternatively, the active agent may be stored in a reservoir such as a porous structure or a gel. Also as discussed in U.S. Pat No. 5,395,310, an ion exchange membrane may be positioned to serve as a polarity selective barrier between the active agent reservoir and the biological interface.

Efficiency and/or timeliness of active agent delivery are of foremost importance when considering the commercial acceptance of iontophoresis devices. For instance, large molecules such as peptides, proteins and nucleic acids (including oligonucleotides) normally do not passively get across the intact mammalian skin. They are therefore likely candidates for iontophoretic delivery as they are typically charged molecules and their transdermal permeation can be actively driven by the electromotive force. However, their delivery across a biological interface is typically limited by their poor bioavailability due to significant degradation by naturally occurring enzymes in the biological tissues beyond the biological interface.

The commercial acceptance of iontophoresis devices is further dependent on a variety of factors, such as cost to manufacture, shelf life or stability during storage, biological capability and/or disposal issues. An iontophoresis device that addresses one or more of the above factors is desirable.

### BRIEF SUMMARY OF THE INVENTION

In one embodiment, an iontophoresis device is provided for the delivery of active agents encapsulated in vesicles such as transferosomes and niosomes to a biological interface such as skin or mucous membranes. The active agent encapsulated in such fashion may have improved bioavailability during and after the delivery. In particular, high molecular weight active agents such as protein or nucleic acid encapsulated in a vesicular carrier may be capable of withstanding enzymatic degradation in the biological tissues. Accordingly, an iontophoresis device as described herein comprise an active electrode element operable to provide an electrical potential; and an inner active agent reservoir comprising a plurality of vesicles, at least some of the vesicles encapsulating an active agent. In certain embodiments, the vesicles are highly deformable transferosomes. In other embodiments, the vesicles are non-ionic niosomes.

Other embodiments describe a method for transdermal administration of an active agent by iontophoresis, the method comprising: positioning an active electrode assembly and a counter electrode assembly of an iontophoresis device on a biological interface of a subject, the active electrode assembly further including an active electrode element operable to provide an electrical potential; and an inner active agent reservoir comprising a plurality of vesicles, at least some of the vesicles encapsulating an active agent; and applying a sufficient amount of current to administer a therapeutically effective amount of the active agent encapsulated in the vesicles in the subject for a limited period of time.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a block diagram of an iontophoresis device comprising active and counter electrode assemblies according to one illustrated embodiment.

FIG. 2 is a block diagram of the iontophoresis device of FIG. 1 positioned on a biological interface, with the outer release liner removed to expose the active agent according to one illustrated embodiment.

### DETAILED DESCRIPTION OF THE INVENTION

The iontophoresis device described herein comprises vesicle-encapsulated active agents housed in an active agent reservoir. In particular, the active agents are incorporated into highly deformable lipid-based vesicles that are capable of passing through natural pores of the stratum corneum under the electrical field. In another embodiment, the active agents can be incorporated into non-ionic surfactant-based vesicles. Advantageously, the iontophoresis device is expected to increase the delivery efficiency of the active agents because they are encapsulated in a protective vesicular carrier. The vesicular carriers are capable of withstanding the enzymatic degradation or other destructive forces that may adversely affect the bioavailability of the active agents. The encapsulated active agents delivered are apt to be released in a sustained and/or controlled manner, preferably at the targeted tissue.

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with controllers including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used herein and in the claims, the term “vesicle” or “vesicular carrier” means a stable structure which is generally characterized by the presence of one or more walls or lamellae which form one or more internal voids or cores. Vesicles may be formulated, for example, from stabilizing compounds, such as a lipid, including the various lipids described herein, a polymer, including the various polymers described herein, or a protein, including the various proteins described herein, as well as using other materials that will be readily apparent to one skilled in the art. Other suitable materials include, for example, any of a wide variety of surfactants, inorganic compounds, and other compounds as will be readily apparent to one skilled in the art. The lipids, polymers, proteins, surfactants, inorganic compounds, and/or other compounds may be natural, synthetic or semi-synthetic. Typically, a stabilizing compound is an amphiphilic compounds having a polar (hydrophilic) head and a lipid (hydrophobic) tail. The polar head group can be ionic or non-ionic. The stabilizing compounds typically can dissolve in aqueous media and self-assemble into vesicles, as defined herein.

The walls or lamellae may be concentric or otherwise. In certain embodiments, the stabilizing compounds form a monolayer or a bilayer. In an aqueous medium, vesicles can take the forms of unilamellar vesicles (comprised of one monolayer or bilayer) or multilamellar vesicles (comprised of two or more monolayers or bilayers). The walls of the vesicles prepared from lipids, polymers or proteins may be substantially solid (uniform), or they may be porous or semi-porous. Regardless of the forms, the wall of a vesicle creates an interface between an organic and an aqueous environment. The voids defined by the wall are capable of entrapping water and water-soluble substances, whereas within the thickness of the walls are aggregates of organic moieties, such as hydrocarbon chains.

The vesicles described herein include such entities commonly referred to as, for example, liposomes, transferosomes, niosomes, micelles, bubbles, microbubbles, microspheres, microballoons, microcapsules, aerogels, clathrate bound vesicles, hexagonal H II phase structures, and the like. The vesicles may also comprise a targeting ligand for targeted delivery.

The vesicles can have net surface charges. Depends on the particular type of stabilizing molecules, the surfaces of the vesicles can be positively or negatively charged. The surface of the vesicles can also be modified to carry charges customized to a specific application. The vesicles can also be electrically neutral.

As used herein, the term “liposomes” means a stable, spherical or spheroidal cluster or aggregate of lipid-based amphiphilic compounds stacked in bilayer arrays. Phospholipids having ionic polar head groups are the most common building blocks for liposomes. Liposomes of this type are typically negatively charged (anionic liposomes) due to presence of the phosphate groups. Liposomes having net positive charges (cationic liposomes) are also well known in the art.

As used herein, the term “transferosomes” refers to a type of highly deformable vesicle. Structurally, transferosomes are analogous to liposome in that transferosomes can also comprise phospholipid-based bilayers. However, transferosomes further comprise cholesterol and additional surfactant molecules such as sodium cholate, the combination of which produce a boundary-active substance typically larger than liposomes or other known similar carrier systems. Significantly, while under pressure, transferosomes are highly deformable and can squeeze through pores one-tenth of their diameters. Transferosomes can be prepared by the methods described in WO 92/03122, WO 98/172500, U.S. patent publication Nos. 2004/0105881, 2002/0048596 and U.S. Pat. No. 6,165,500, all of which are incorporated herein by reference in their entireties. Transferosomes are also commercially available under the registered trade name “Transfersome®” from IDEA AG (Munich, Germany).

In one embodiment, the iontophoresis device described herein employs transferosomes as a carrier to transport an active agent across a biological interface. The biological interface, such as skin, is expected to be particularly permeable with respect to the transferosome carriers due to their flexible and deformable nature. Like liposomes, the surface of the transferosomes can be charged or electrically neutral. The cores of the transferosomes are typically water-filled and can encapsulate any water-soluble substance, including an active agent.

Generally speaking, during iontophoresis, charged or uncharged species (including active agents and vesicles) can migrate across a permeable biological interface into the underlying biological tissue. Typically, an iontophoresis device generates both electro-repulsive and electro-osmotic forces. For charged species, the migration is primarily driven by electro-repulsion between the oppositely charged active electrode and the charged species. In addition to the electro-repulsive forces, the electro-osmotic flow of a liquid (e.g., a solvent or diluent) may also contribute to transporting the charged species. In certain embodiments, the electro-osmotic solvent flow is a secondary force that can enhance the migration of the charged species. For uncharged or neutral species, the migration is primarily driven by the electro-osmotic flow of a solvent.

Accordingly, transferosomes can be transported across a permeable biological interface under the electro-repulsive and electro-osmotic forces generated by the iontophoresis device. Typically, enhanced migration via electro-osmotic solvent flow may be more pronounced with the increasing sizes of the molecules transported. In one embodiment, large molecules, including protein and/or nucleic acid-based active agents that are susceptible to enzymatic degradation in biological tissues can be encapsulated in the transferosomes and transported across the permeable biological interface using the iontophoresis device described herein.

In another embodiment, niosomes can be used as an alternative vesicular carrier of an active agent. “Niosomes” are non-ionic surfactant vesicle. Classes of commonly used non-ionic surfactants include polyglycerol alkylethers, glucosyl dialkylethers, crown ethers and polyoxyethylene alkyl ethers and esters. Examples of niosomes include, but are not limited to, polyoxyethylene fatty acid esters, polyoxyethylene fatty alcohols, polyoxyethylene fatty alcohol ethers, polyoxyethylated sorbitan fatty acid esters, glycerol polyethylene glycol oxystearate, glycerol polyethylene glycol ricinoleate, ethoxylated soybean sterols, ethoxylated castor oil, polyoxyethylene-polyoxypropylene polymers, and polyoxyethylene fatty acid stearates; sterol aliphatic acid esters including cholesterol sulfate, cholesterol butyrate, cholesterol iso-butyrate, cholesterol palmitate, cholesterol stearate, lanosterol acetate, ergosterol palmitate, and phytosterol n-butyrate; sterol esters of sugar acids including cholesterol glucuronide, lanosterol glucuronide, 7-dehydrocholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl gluconate; esters of sugars and aliphatic acids including sucrose laurate, fructose laurate, sucrose palmitate, sucrose stearate, glucuronic acid, gluconic acid, accharic acid, and polyuronic acid; saponins including sarsasapogenin, smilagenin, hederagenin, oleanolic acid, and digitoxigenin; glycerol dilaurate, glycerol trilaurate, glycerol dipalmitate, glycerol, and glycerol esters including glycerol tripalmitate, glycerol distearate, glycerol tristearate, glycerol dimyristate, and glycerol trimyristate; longchain alcohols including n-decyl alcohol, lauryl alcohol, myristyl alcohol, cetyl alcohol, and n-octadecyl alcohol; 1,2-dioleoyl-sn-glycerol; 1,2-dipalmitoyl-sn-3-succinylglycerol; 1,3-dipalmitoyl-2-succinylglycerol; 1-hexadecyl-2-palmitoylglycerophosphoe thanolamine and palmitoylhomocysteine; and/or combinations thereof. Some of the surfactants are commercially available under the trade names of TWEEN™ and SPAN™.

“Active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., a drug, a therapeutic compound, pharmaceutical salts, and the like) non-pharmaceuticals (e.g., cosmetic substance, and the like), a vaccine, an immunological agent, a local or general anesthetic or painkiller, an antigen or a protein or peptide such as insulin, a chemotherapy agent, an anti-tumor agent.

In some embodiments, the term “active agent” further refers to the active agent, as well as its pharmacologically active salts, pharmaceutically acceptable salts, prodrugs, metabolites, analogs, and the like. In some further embodiment, the active agent includes at least one ionic, cationic, anionic, ionizable, and/or neutral therapeutic drug and/or pharmaceutical acceptable salts thereof.

In certain embodiments, the active agent may be charged, may have a low net charge, or may be uncharged (electrically neutral) under the conditions within the active electrode.

In certain embodiments, the active agent may be a high molecular weight molecule (i.e., higher than 1,000 Daltons). In certain other embodiments, the molecule may be a polar polyelectrolyte. In certain other embodiments, the molecule may be lipophilic. In certain embodiments, the high molecular weight polyelectrolytic active agents may be proteins, polypeptides or nucleic acids. Specific examples of high molecular weight active agents typically include, but are not limited to: DNA, insulin-like growth factors (IGF), bone morphogenetic proteins (BMP), heparin-binding fibroblast growth factor (FGF), platelet-derived growth factors (PDGF), TGF-β, parathyroid hormone (PTH), and statins.

Certain high molecular weight active agents are prone to be degraded or otherwise adversely affected by the numerous subcutaneous enzymes. The vesicles protect the entrapped active agents from the enzymatic environment, hence increasing their bioavailability.

The active agents suitable for the iontophoresis device described herein can be selected largely based on the efficiency of their entrapment within the vesicles. Typically, active agents that are water-soluble are likely to be entrapped within the internal core of the vesicles. To that end, reverse phase evaporation (REV) can be employed to entrap active agents in the vesicles; such technology is within the knowledge of one skilled in the art. The efficiency of the entrapment of active agents in the vesicles (transferosomes or niosomes) are determinable using known technologies, such as gel filtration, transmission electron microscopy or high-resolution optical microscopy. In one embodiment, at least 10% of the vesicles contain encapsulated active agent. In another embodiment, at least 30% of the vesicles contain encapsulated active agent. In a further embodiment, at least 60% of the vesicles contain encapsulated active agent.

The iontophoretic delivery of encapsulated active agent can further effect controlled release of the entrapped active agent. “Controlled release”, as used herein, means a method and composition for making an active agent available to the biological system of a host. Controlled-release includes the use of instantaneous release, delayed release, and sustained release. “Instantaneous release” refers to immediate release to the biosystem of the host. “Delayed release” means the active ingredient is not made available to the host until some time delay after administration. “Sustained Release” generally refers to release of active agents whereby the level of active agent available to the host is maintained at some level over a period of time. Advantageously, active agents encapsulated in a vesicular carrier delivered into biological tissues or circulation using iontophoresis can be released in a controlled manner. For instance, a tumor site may be associated with a higher temperature or different pH from that of the circulation. These external factors may cause the vesicles to leak or rupture, resulting in the controlled release of the active agent locally. Additionally, some tumor sites preferentially take up lipid-based vesicles, thereby releasing the active agent entrapped therein.

As used herein and in the claims, “bioavailability” refers to the rate and relative amount of the administered active agent which reaches the general circulation intact.

The bioavailability of certain active agents can be expected to increase due to the protective function of the vesicles. Thus, in a further embodiment, a method for transdermal administration of an active agent by iontophoresis is the described. The method comprising: positioning an active electrode assembly and a counter electrode assembly of an iontophoresis device on a biological interface of a subject, the active electrode assembly further including an active electrode element operable to provide an electrical potential; and an inner active agent reservoir comprising a plurality of vesicles, at least some of the vesicles encapsulating an active agent; and applying a sufficient amount of current to administer a therapeutically effective amount of the active agent encapsulated in the vesicles in the subject for a limited period of time.

In a further embodiment, the method comprises releasing the active agent from the vesicles, following the transdermal administration. Depending on the compositions of the vesicles and the triggering event that may rupture or cause the formation of openings in the vesicles, the entrapped active agents can be released accordingly to different profiles. Typically, the release profile of the active agent may be a controlled release or sustained release. In a preferred embodiment, the release of the active agent is triggered by a targeted treatment site, for instance, a tumor site.

As used herein and in the claims, the term “membrane” means a layer, barrier or material, which may, or may not be permeable. Unless specified otherwise, membranes may take the form a solid, liquid or gel, and may or may not have a distinct lattice or cross-linked structure.

As used herein and in the claims, the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein and in the claims, the term “ion selective membrane” or “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane permits only the passage of cations and substantially blocks anions. Examples of commercially available cation exchange membranes include those available under the designators NEOSEPTA, CM-1, CM-2, CMX, CMS, and CMB from Tokuyama Co., Ltd. Conversely, An anion exchange membrane permits only the passage of anions and substantially blocks cations. Examples of commercially available anion exchange membranes include those available under the designators NEOSEPTA, AM-1, AM-3, AMX, AHA, ACH and ACS also from Tokuyama Co., Ltd.

As used herein and in the claims, term “bipolar membrane” means a membrane that is selective to two different charges or polarities. Unless specified otherwise, a bipolar membrane may take the form of a unitary membrane structure or multiple membrane structure. The unitary membrane structure may having a first portion including cation ion exchange material or groups and a second portion opposed to the first portion, including anion ion exchange material or groups. The multiple membrane structure (e.g., two film) may be formed by a cation exchange membrane attached or coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that substantially selective based on a size or molecular weight of the ion. Thus, a semi-permeable membrane substantially passes ions of a first molecular weight or size, while substantially blocking passage of ions of a second molecular weight or size, greater than the first molecular weight or size.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “reservoir” means any form of mechanism to retain an element or compound in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force into the biological interface. A reservoir may also retain an electrolyte solution.

The headings provided herein are for convenience only and do not interpret the scope or meaning of the embodiments.

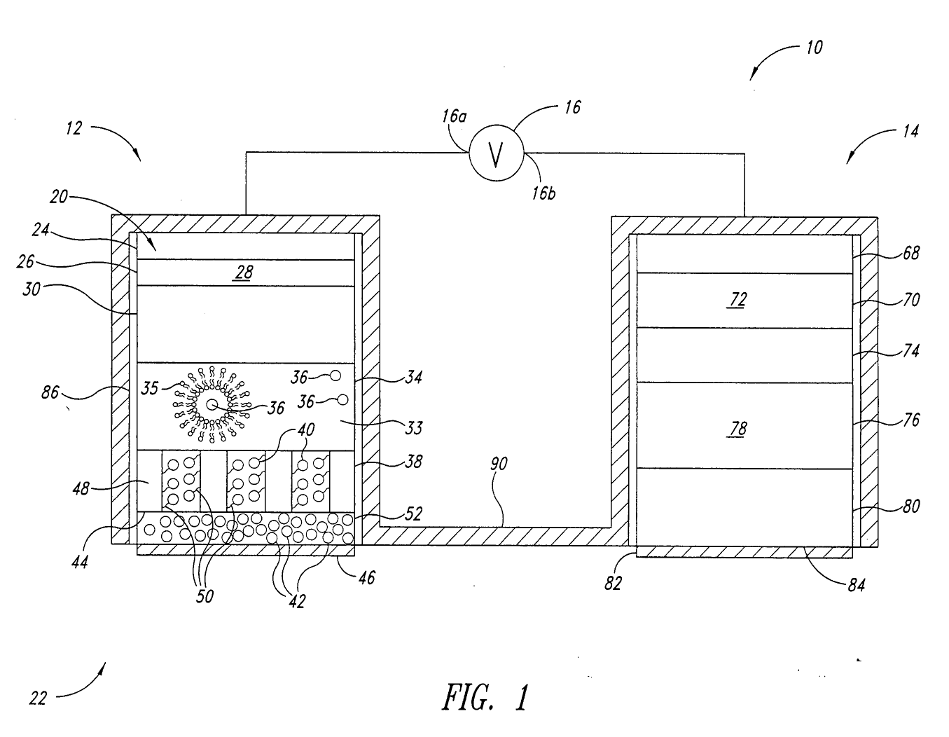

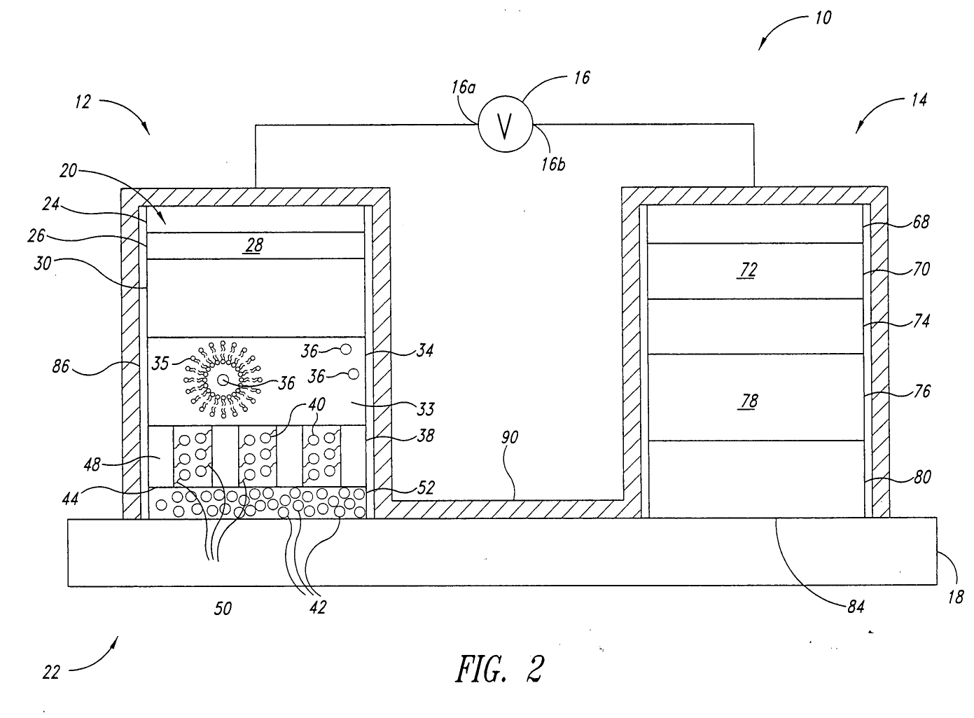

FIGS. 1 and 2 show an iontophoresis device 10 comprising active and counter electrode assemblies, 12, 14, respectively, electrically coupled to a power source 16, operable to supply an active agent contained in the active electrode assembly 12 to a biological interface 18 (FIG. 2), such as a portion of skin or mucous membrane via iontophoresis, according to one illustrated embodiment.

In the illustrated embodiment, the active electrode assembly 12 comprises, from an interior 20 to an exterior 22 of the active electrode assembly 12: an active electrode element 24, an optional electrolyte reservoir 26 storing an electrolyte 28, an optional inner ion selective membrane 30, an inner active agent reservoir 34 storing an active agent 36 encapsulated in a vesicle 35 (only one shown), an optional outermost ion selective membrane 38 that optionally caches additional active agent 40, an optional further active agent 42 carried by an outer surface 44 of the outermost ion selective membrane 38, and an outer release liner 46. Unless specified otherwise, active agents 36, 40 and 42, when referred below, are considered to be in either encapsulated or free form. Each of the above elements or structures will be discussed in detail below.

The active electrode element 24 is coupled to a first pole 16a of the voltage source 16 and positioned in the active electrode assembly 12 to apply an electromotive force or current to transport encapsulated active agent 36, 40, 42 via various other components of the active electrode assembly 12. The active electrode element 24 may take a variety of forms. For example, the active electrode element 24 may include a,sacrificial element such as a chemical compound or amalgam including silver (Ag) or silver chloride (AgCl). Such compounds or amalgams typically employ one or more heavy metals, for example lead (Pb), which may present issues with regard manufacturing, storage, use and/or disposal. Consequently, some embodiments may advantageously employ a non-metallic active electrode element. Such may, for example, comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004.

The active electrode assembly 12 may optionally comprise an electrolyte reservoir 26 positioned between the active electrode element 24 and the outermost active electrode ion selective membrane 38, proximate the active electrode element 24. The electrolyte 28 contained therein may provide ions or donate charges to prevent or inhibit the formation of gas bubbles (e.g., hydrogen) on the active electrode element 24 in order to enhance efficiency and/or increase delivery rates. This elimination or reduction in electrolysis may in turn inhibit or reduce the formation of acids and/or bases (e.g., H⁺ions, OH⁻ions), that would otherwise present possible disadvantages such as reduced efficiency, reduced transfer rate, and/or possible irritation of the biological interface 18. As discussed in further below, in some embodiments the electrolyte 28 may provide or donate ions to substitute for the active agent in the outermost ion selective membrane 38, for example substituting for the active agent 40 bonded to the ion exchange groups 50 where the outermost active electrode ion selective membrane 38 takes the form of an ion exchange membrane. Such may facilitate transfer of the active agent 40 to the biological interface 18, for example, increasing and/or stabilizing delivery rates. A suitable electrolyte may take the form of a solution of 0.5M disodium fumarate:0.5M poly(acrylic acid) (5:1).

Optionally, an inner ion selective membrane 30 can be positioned to separate the electrolyte 28 and the inner active agent reservoir 34. The inner ion selective membrane 30 may take the form of a charge selective membrane. For example, if the vesicular carriers 35 of the active agent 36 are positively charged, the inner ion selective membrane 30 may take the form of an anion exchange membrane, selective to substantially pass anions and substantially block cations. The inner ion selective membrane 30 may advantageously prevent transfer of undesirable elements or compounds between the electrolyte 28 and the inner active agent reservoir 34. For example, the inner ion selective membrane 30 may prevent or inhibit the transfer of sodium (Na⁺), proton (H⁺) ions from the electrolyte 28, thereby increases the transfer rate and/or biological compatibility of the iontophoresis device 10.

The inner active agent reservoir 34 is generally positioned between the inner ion selective membrane 30 and the outermost ion selective membrane 38, if such ion selective membranes are to be employed. The inner active agent reservoir 34 may take a variety of forms including any structure capable of temporarily retaining active agent 36. For example, the inner active agent reservoir 34 may take the form of a pouch or other receptacle, a membrane with pores, cavities or interstices, particularly where the active agent 36 is a liquid. The inner active agent reservoir 34 may advantageously allow larger doses of the active agent 36 to be loaded in the active electrode assembly 12. Regardless of the form, the inner active agent reservoir 34 allows for the loading of a solution or dispersion of the vesicle 35-encapsulated active agents 36. (Only one such vesicle is shown for sake of clarity of illustration.)

Optionally, an outermost ion selective membrane 38 can be positioned generally opposed across the active electrode assembly 12 from the active electrode element 24. The outermost membrane 38 may, as in the embodiment illustrated in FIGS. 1 and 2, take the form of an ion exchange membrane, pores 48 (only one called out in FIGS. 1 and 2 for sake of clarity of illustration) of the ion selective membrane 38 including ion exchange material or groups 50 (only three called out in FIGS. 1 and 2 for sake of clarity of illustration). Under the influence of an electromotive force or current, the ion exchange material or groups 50 selectively substantially passes ions of the same polarity as vesicles encapsulating active agent 36, 40, respectively, while substantially blocking ions of the opposite polarity. Thus, the outermost ion exchange membrane 38 is charge selective. Where the vesicles 35 entrapping active agent 36 are cationic, the outermost ion selective membrane 38 may take the form of a cation exchange membrane. Alternatively, where the vesicles 35 are anionic, the outermost ion selective membrane 38 may take the form of an anion exchange membrane. As noted above, the vesicles described herein may also be electrically neutral, in which case, the ion exchange membranes may nonetheless employed to block the undesirable ions from entering the inner active agent reservoir which may impede the transportation of the active agent.

The outermost ion selective membrane 38 may advantageously cache active agent 40, optionally encapsulated in vesicles. In particular, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

The outermost ion selective membrane 38 may also be preloaded with the additional active agent 40, optionally encapsulated in vesicle. Where the outermost ion selective membrane 38 is an ion exchange membrane, a substantial amount of active agent 40 may bond to ion exchange groups 50 in the pores, cavities or interstices 48 of the outermost ion selective membrane 38.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically, vapor deposition, and/or otherwise. In some embodiments, the further active agent 42 may sufficiently cover the outer surface 44 and/or be of sufficient thickness so as to form a distinct layer 52. In other embodiments, the further active agent 42 may not be sufficient in volume, thickness or coverage as to constitute a layer in a conventional sense of such term.

The active agent 42, whether in encapsulated or in free form, may be deposited in a variety of highly concentrated forms such as, for example, solid form, nearly saturated solution form or gel form. If in solid form, a source of hydration may be provided, either integrated into the active electrode assembly 12, or applied from the exterior thereof just prior to use. If the active agent 42 is pre-encapsulated in vesicles, the solid form should be capable of forming a dispersion of individual vesicles upon hydration.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. In some embodiment, one or all of the active agents 36, 40 and 42 are encapsulated in vesicles, with the proviso that at least one of them is encapsulated in vesicles. Thus, a first type of active agent may be stored in the inner active agent reservoir 34, while a second type of active agent may be cached in the outermost ion selective membrane 38. In such an embodiment, either the first type or the second type of active agent may be deposited on the outer surface 44 of the outermost ion selective membrane 38 as the further active agent 42. Alternatively, a mix of the first and the second types of active agent may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first type of active agent may be stored in the inner active agent reservoir 34 as the active agent 36 and cached in the outermost ion selective membrane 38 as the additional active agent 40, while a second type of active agent may be deposited on the outer surface 44 of the outermost ion selective membrane 38 as the further active agent 42. Typically, in embodiments where one or more different active agents are employed, the active agents 36, 40 and 42 will all be of common polarity to prevent the active agents 36, 40 and 42 from competing with one another. Other combinations are possible.

The outer release liner 46 may generally be positioned overlying or covering further active agent 42 carried by the outer surface 44 of the outermost ion selective membrane 38. The outer release liner 46 may protect the further active agent 42 and/or outermost ion selective membrane 38 during storage, prior to application of an electromotive force or current. The outer release liner 46 may be a selectively releasable liner made of waterproof material, such as release liners commonly associated with pressure sensitive adhesives. Note that the inner release liner 46 is shown in place in FIG. 1 and removed in FIG. 2.

An interface-coupling medium (not shown) may be employed between the electrode assembly and the biological interface 18. The interface-coupling medium may, for example, take the form of an adhesive and/or gel. The gel may, for example, take the form of a hydrating gel.

In the embodiment illustrated in FIGS. 1 and 2, the counter electrode assembly comprises, in order to form an interior 64 to an exterior 66 of the counter electrode assembly 14: a counter electrode element 68, electrolyte reservoir 70 storing an electrolyte 72, an inner ion selective membrane 74, an optional buffer reservoir 76 storing buffer material 78, an optional outermost ion selective membrane 80, and an optional outer release liner 82.

The counter electrode element 68 is electrically coupled to a second pole 16b of the voltage source 16, the second pole 16b having an opposite polarity to the first pole 16a. The counter electrode element 68 may take a variety of forms. For example, the counter electrode element 68 may include a sacrificial element, such as a chemical compound or amalgam including silver (Ag) or silver chloride (AgCl), or may include a non-sacrificial element such as the carbon-based electrode element discussed above.

The electrolyte reservoir 70 may take a variety of forms including any structure capable of retaining electrolyte 72, and in some embodiments may even be the electrolyte 72 itself, for example, where the electrolyte 72 is in a gel, semi-solid or solid form. For example, the electrolyte reservoir 70 may take the form of a pouch or other receptacle, or a membrane with pores, cavities or interstices, particularly where the electrolyte 72 is a liquid.

The electrolyte 72 is generally positioned between the counter electrode element 68 and the outermost ion selective membrane 80, proximate the counter electrode element 68. As described above, the electrolyte 72 may provide ions or donate charges to prevent or inhibit the formation of gas bubbles (e.g., hydrogen) on the counter electrode element 68 and may prevent or inhibit the formation of acids or bases or neutralize the same, which may enhance efficiency and/or reduce the potential for irritation of the biological interface 18.

The inner ion selective membrane 74 is positioned between and/or to separate, the electrolyte 72 from the buffer material 78. The inner ion selective membrane 74 may take the form of a charge selective membrane, such as the illustrated ion exchange membrane that substantially allows passage of ions of a first polarity or charge while substantially blocking passage of ions or charge of a second, opposite polarity. The inner ion selective membrane 74 will typically pass ions of opposite polarity or charge to those passed by the outermost ion selective membrane 80 while substantially blocking ions of like polarity or charge. Alternatively, the inner ion selective membrane 74 may take the form of a semi-permeable or microporous membrane that is selective based on size.

The inner ion selective membrane 74 may prevent transfer of undesirable elements or compounds into the buffer material 78. For example, the inner ion selective membrane 74 may prevent or inhibit the transfer of hydroxy (OH⁻) or chloride (Cl⁻) ions from the electrolyte 72 into the buffer material 78.

The optional buffer reservoir 76 is generally disposed between the electrolyte reservoir and the outermost ion selective membrane 80. The buffer reservoir 76 may take a variety of forms capable of temporarily retaining the buffer material 78. For example, the buffer reservoir 76 may take the form of a cavity, a porous membrane or a gel.

The buffer material 78 may supply ions for transfer through the outermost ion selective membrane 42 to the biological interface 18. Consequently, the buffer material 78 may, for example, comprise a salt (e.g., NaCl).

The outermost ion selective membrane 80 of the counter electrode assembly 14 may take a variety of forms. For example, the outermost ion selective membrane 80 may take the form of a charge selective ion exchange membrane, such as a cation exchange membrane or an anion exchange membrane, which substantially passes and/or blocks ions based on the charge carried by the ion. Examples of suitable ion exchange membranes are discussed above. Alternatively, the outermost ion selective membrane 80 may take the form of a semi-permeable membrane that substantially passes and/or blocks ions based on size or molecular weight of the ion.

The outermost ion selective membrane 80 of the counter electrode assembly 14 is selective to ions with a charge or polarity opposite to that of the outermost ion selective membrane 38 of the active electrode assembly 12. Thus, for example, where the outermost ion selective membrane 38 of the active electrode assembly 12 allows passage of negatively charged ions of the active agent 36, 40, 42 to the biological interface 18, the outermost ion selective membrane 80 of the counter electrode assembly 14 allows passage of positively charged ions to the biological interface 18, while substantially blocking passage of ions having a negative charge or polarity. On the other hand, where the outermost ion selective membrane 38 of the active electrode assembly 12 allows passage of positively charged ions of the active agent 36, 40 ,42 to the biological interface 18, the outermost ion selective membrane 80 of the counter electrode assembly 14 allows passage of negatively charged ions to the biological interface 18 while substantially blocking passage of ions with a positive charge or polarity.

The outer release liner 82 may generally be positioned overlying or covering an outer surface 84 of the outermost ion selective membrane 80. Note that the inner release liner 82 is shown in place in FIG. 1 and removed in FIG. 2. The outer release liner 82 may protect the outermost ion selective membrane 80 during storage, prior to application of an electromotive force or current. The outer release liner 82 may be a selectively releasable liner made of waterproof material, such as release liners commonly associated with pressure sensitive adhesives. In some embodiments, the outer release liner 82 may be coextensive with the outer release liner 46 of the active electrode assembly 12.

The power source 16 may take the form of one or more chemical battery cells, super—or highly-capacitors, or fuel cells. The power source 16 may, for example, provide a voltage of 12.8 V DC, with tolerance of 0.8V DC, and a current of 0.3 mA. The power source 16 may be selectively electrically coupled to the active and counter electrode assemblies 12a, 14 via a control circuit, for example, via carbon fiber ribbons. The iontophoresis device 10 may include discrete and/or integrated circuit elements to control the voltage, current and/or power delivered to the electrode assemblies 12, 14. For example, the iontophoresis device 10 may include a diode to provide a constant current to the electrode elements 20, 40.

As suggested above, the vesicles 35, 43 and 45 of the respective active agent 36, 40, 42 may take be cationic or anionic. Consequently, the terminals or poles 16a, 16b of the voltage source 16 may be reversed. Likewise, the selectivity of the outermost ion selective membranes 38, 80 and inner ion selective membranes 30, 74 may be reversed.

The iontophoresis device 10 may further comprise an inert molding material 86 adjacent exposed sides of the various other structures forming the active and counter electrode assemblies 12, 14. The molding material 86 may advantageously provide environmental protection to the various structures of the active and counter electrode assemblies 12, 14.

As best seen in FIG. 2, the active and counter electrode assemblies 12, 14 are positioned on the biological interface 18. Positioning on the biological interface may close the circuit, allowing electromotive force to be applied and/or current to flow from one pole 16a of the voltage source 16 to the other pole 16b, via the active electrode assembly, biological interface 18 and counter electrode assembly 14.

In the presence of the electromotive force and/or current, active agent 36 encapsulated in vesicles (such as transferosomes or niosomes) are transported toward the biological interface 18. Additional active agent 40 is released by the ion exchange groups or material 50 by the substitution of ions of the same charge or polarity (e.g., active agent 36), and transported toward the biological interface 18. While some of the active agent 36 may substitute for the additional active agent 40, some of the active agent 36 may be transferred through the outermost ion elective membrane 38 into the biological interface 18. Further active agent 42 carried by the outer surface 44 of the outermost ion elective membrane 38 is also transferred to the biological interface 18.

The above description of illustrated embodiments, including what is described in the Abstract, is not intended to be exhaustive or to limit the claims to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to other agent delivery systems and devices, not necessarily the exemplary iontophoresis active agent system and devices generally described above. For instance, some embodiments may omit some of the reservoirs, membranes or other structures. For example, some embodiment may include a control circuit or subsystem to control a voltage, current or power applied to the active and counter electrode elements 20, 40. Also for example, some embodiments may include an interface layer interposed between the outermost active electrode ion selective membrane 38 and the biological interface 18. Some embodiments may comprise additional ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. No. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240,995, herein incorporated in their entirety by reference. Further examples of such hydrogels are disclosed in U.S. patent applications 2004/166147; 2004/105834; and 2004/247655, herein incorporated in their entirety by reference. Product brand names of various hydrogels and hydrogel sheets include Corplex™ by Corium, Tegagel™ by 3M, PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd.

The various embodiments discussed above may advantageously employ various microstructures, for example microneedles. Microneedles and microneedle arrays, their manufacture, and use have been described. Microneedles, either individually or in arrays, may be hollow; solid and permeable; solid and semi-permeable; or solid and non-permeable. Solid, non-permeable microneedles may further comprise grooves along their outer surfaces. Microneedle arrays, comprising a plurality of microneedles, may be arranged in a variety of configurations, for example rectangular or circular. Microneedles and microneedle arrays may be manufactured from a variety of materials, including silicon; silicon dioxide; molded plastic materials, including biodegradable or non-biodegradable polymers; ceramics; and metals. Microneedles, either individually or in arrays, may be used to dispense or sample fluids through the hollow apertures, through the solid permeable or semi-permeable materials, or via the external grooves. Microneedle devices are used, for example, to deliver a variety of compounds and compositions to the living body via a biological interface, such as skin or mucous membrane. In certain embodiments, the active agent compounds and compositions may be delivered into or through the biological interface. For example, in delivering compounds or compositions via the skin, the length of the microneedle(s), either individually or in arrays, and/or the depth of insertion may be used to control whether administration of a compound or composition is only into the epidermis, through the epidermis to the dermis, or subcutaneous. In certain embodiments, microneedle devices may be useful for delivery of high-molecular weight active agents, such as those comprising proteins, peptides and/or nucleic acids, and corresponding compositions thereof. In certain embodiments, for example wherein the fluid is an ionic solution, microneedle(s) or microneedle array(s) can provide electrical continuity between a power source and the tip of the microneedle(s). Microneedle(s) or microneedle array(s) may be used advantageously to deliver or sample compounds or compositions by iontophoretic methods, as disclosed herein. In certain embodiments, for example, a plurality of microneedles in an array may advantageously be formed on an outermost biological interface-contacting surface of an iontophoresis device. Compounds or compositions delivered or sampled by such a device may comprise, for example, high-molecular weight active agents, such as proteins, peptides and/or nucleic acids.

In certain embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; an active agent reservoir having an active agent solution that is in contact with the first electrode member and to which is applied a voltage via the first electrode member; a biological interface contact member, which may be a microneedle array and is placed against the forward surface of the active agent reservoir; and a first cover or container that accommodates these members. The counter electrode assembly includes the following: a second electrode member connected to a negative electrode of the power source; an electrolyte reservoir that holds an electrolyte that is in contact with the second electrode member and to which voltage is applied via the second electrode member; and a second cover or container that accommodates these members.

In certain other embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; a first electrolyte reservoir having an electrolyte that is in contact with the first electrode member and to which is applied a voltage via the first electrode member; a first anion-exchange membrane that is placed on the forward surface of the first electrolyte reservoir; an active agent reservoir that is placed against the forward surface of the first anion-exchange membrane; a biological interface contacting member, which may be a microneedle array and is placed against the forward surface of the active agent reservoir; and a first cover or container that accommodates these members. The counter electrode assembly includes the following: a second electrode member connected to a negative electrode of the power source; a second electrolyte reservoir having an electrolyte that is in contact with the second electrode member and to which is applied a voltage via the second electrode member; a cation-exchange membrane that is placed on the forward surface of the second electrolyte reservoir; a third electrolyte reservoir that is placed against the forward surface of the cation-exchange membrane and holds an electrolyte to which a voltage is applied from the second electrode member via the second electrolyte reservoir and the cation-exchange membrane; a second anion-exchange membrane placed against the forward surface of the third electrolyte reservoir; and a second cover or container that accommodates these members.

Certain details of microneedle devices, their use and manufacture, are disclosed in U.S. Pat. Nos. 6,256,533; 6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; 6,939,311; all of which are incorporated herein by reference in their entirety. Some or all of the teaching therein may be applied to microneedle devices, their manufacture, and their use in iontophoretic applications.

Aspects of the various embodiments can be modified, if necessary, to employ systems, circuits and concepts of the various patents, applications and publications to provide yet further embodiments, including those patents and applications identified herein. While some embodiments may include all of the membranes, reservoirs and other structures discussed above, other embodiments may omit some of the membranes, reservoirs or other structures. Still other embodiments may employ additional ones of the membranes, reservoirs and structures generally described above. Even further embodiments may omit some of the membranes, reservoirs and structures described above while employing additional ones of the membranes, reservoirs and structures generally described above.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: Japanese patent application Serial No. H03-86002, filed Mar. 27, 1991, having Japanese Publication No. H04-297277, issued on Mar. 3, 2000 as Japanese Patent No. 3040517; Japanese patent application Serial No. 11-033076, filed Feb. 10, 1999, having Japanese Publication No. 2000-229128; Japanese patent application Serial No. 11-033765, filed Feb. 12, 1999, having Japanese Publication No. 2000-229129; Japanese patent application Serial No. 11-041415, filed Feb. 19, 1999, having Japanese Publication No. 2000-237326; Japanese patent application Serial No. 11-041416, filed Feb. 19, 1999, having Japanese Publication No. 2000-237327; Japanese patent application Serial No. 11-042752, filed Feb. 22, 1999, having Japanese Publication No. 2000-237328; Japanese patent application Serial No. 11-042753, filed Feb. 22, 1999, having Japanese Publication No. 2000-237329; Japanese patent application Serial No. 11-099008, filed Apr. 6, 1999, having Japanese Publication No. 2000-288098; Japanese patent application Serial No. 11-099009, filed Apr. 6, 1999, having Japanese Publication No. 2000-288097; PCT patent application WO 2002JP4696, filed May 15, 2002, having PCT Publication No WO03037425; U.S. patent application Ser. No. 10/488970, filed Mar. 9, 2004; U.S. Provisional Patent Application No. 60/722,298, filed Sep. 30, 2005; Japanese patent application 2004/317317, filed Oct. 29, 2004; U.S. provisional patent application Ser. No. 60/627,952, filed Nov. 16, 2004; Japanese patent application Serial No. 2004-347814, filed Nov. 30, 2004; Japanese patent application Serial No. 2004-357313, filed Dec. 9, 2004; Japanese patent application Serial No. 2005-027748, filed Feb. 3, 2005; and Japanese patent application Serial No. 2005-081220, filed Mar. 22, 2005.

Aspects of the various embodiments can be modified, if necessary, to employ systems, circuits and concepts of the various patents, applications and publications to provide yet further embodiments. While some embodiments may include all of the membranes, reservoirs and other structures discussed above, other embodiments may omit some of the membranes, reservoirs or other structures. Still other embodiments may employ additional ones of the membranes, reservoirs and structures generally described above. Even further embodiments may omit some of the membranes, reservoirs and structures described above while employing additional ones of the membranes, reservoirs and structures generally described above.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

We/I claim:

1. An iontophoresis device for delivering active agents to a biological interface, the iontophoresis device comprising an active electrode assembly and a counter electrode assembly, the active electrode assembly further including:
an active electrode element operable to provide an electrical potential; and
an inner active agent reservoir comprising a plurality of first vesicles, at least some of the first vesicles encapsulating a first active agent.

2. The iontophoresis device of claim 1 wherein the first vesicle is a transferosome.

3. The iontophoresis device of claim 2 wherein the transferosome comprises lipids.

4. The iontophoresis device of claim 3 wherein the transferosome further comprises cholesterol, sodium cholate or a combination thereof.

5. The iontophoresis device of claim 2 wherein the transferosome carries a net charge on a surface of the transferosome.

6. The iontophoresis device of claim 1 wherein the first vesicle is a niosome.

7. The iontophoresis device of claim 6 wherein the niosome carries a net charge on a surface of the niosome.

8. The iontophoresis device of claim 3 wherein the niosome is electrically neutral.

9. The iontophoresis device of claim 1 wherein as least 10% of the first vesicles contain first active agent.

10. The iontophoresis device of claim 1 wherein as least 30% of the first vesicles contain first active agent.

11. The iontophoresis device of claim 1 wherein as least 60% of the first vesicles contain first active agent.

12. The iontophoresis device of claim 1 further comprising:
an electrolyte reservoir comprising an electrolyte composition; and
an inner ion selective membrane positioned between said electrolyte reservoir and said inner active agent reservoir.

13. The iontophoresis device of claim 12, further comprising:
an outermost ion selective membrane having an outer surface, the outer surface being proximate the biological interface when in use.

14. The iontophoresis device of claim 12, further comprising:
a second active agent cached in the outermost ion selective membrane.

15. The iontophoresis device of claim 14 wherein the second active agent is encapsulated in a second vesicle.

16. The iontophoresis device of claim 12, further comprising:
a third active agent deposited on the out surface of the outermost ion selective membrane.

17. The iontophoresis device of claim 16 wherein the third active agent is encapsulated in a third vesicle.

18. The iontophoresis device of claim 1 wherein the first active agent has a molecular weight higher than 1,000 Daltons.

19. The iontophoresis device of claim 1 wherein the first active agent is DNA, insulin-like growth factors (IGF), bone morphogenetic proteins (BMP), heparin-binding fibroblast growth factor (FGF), platelet-derived growth factors (PDGF), TGF-β, parathyroid hormone (PTH), or statins.

20. A method for transdermal administration of an active agent by iontophoresis, comprising:
positioning an active electrode assembly and a counter electrode assembly of an iontophoresis device on a biological interface of a subject, the active electrode assembly further including an active electrode element operable to provide an electrical potential; and an inner active agent reservoir comprising a plurality of vesicles, at least some of the vesicles encapsulating an active agent; and
applying a sufficient amount of current to administer a therapeutically effective amount of the active agent encapsulated in the vesicles in the subject for a limited period of time.

21. The method of claim 20 wherein the vesicle is a transferosome.

22. The method of claim 21 wherein the transferosome comprises lipids.

23. The method of claim 22 wherein the transferosome further comprises cholesterol, sodium cholate or a combination thereof.

24. The method of claim 20 wherein the vesicle is a niosome.

25. The method of claim 20 wherein the vesicle carries a net charge on a surface thereof.

26. The method of claim 20 wherein the vesicles are electrically neutral.

27. The method of claim 20 wherein the vesicles remain intact during the transdermal administration.

28. The method of claim 20 wherein the active agent is DNA, insulin-like growth factors (IGF), bone morphogenetic proteins (BMP), heparin-binding fibroblast growth factor (FGF), platelet-derived growth factors (PDGF), TGF-β, parathyroid hormone (PTH), or statins.
